SEQUENCE LISTING (4 sequences)

SEQ ID NO: 1 - protein, 9 aa, Homo sapiens
EEQYNSTYR

SEQ ID NO: 2 - protein, 9 aa, Homo sapiens
EEQFNSTFR

SEQ ID NO: 3 - protein, 9 aa, Homo sapiens
EEQYNSTFR

SEQ ID NO: 4 - protein, 9 aa, Homo sapiens
EEQFNSTYR

(19) United States
(12) Patent Application Publication
(10) Pub. No.: US 2023/0417762 A1
(43) Pub. Date: Dec. 28, 2023

(54) SIALYLATED GLYCOPROTEINS

(71) Applicant: Momenta Pharmaceuticals, Inc., Titusville, NJ (US)

(72) Inventors: Nathaniel J. WASHBURN, Titusville, NJ (US); Daniel ORTIZ, Titusville, NJ (US); John SCHAECK, Titusville, NJ (US)

(21) Appl. No.: 18/037,378

(22) PCT Filed: Nov. 19, 2021
(86) PCT No.: PCT/US2021/060182

§ 371 Date: May 17, 2023

Related U.S. Application Data
(60) Provisional application No. 63/116,643, filed on Nov. 20, 2020.

(51) Int. Cl. G01N 33/68

(52) U.S. Cl. CPC G01N 33/6857; G01N 33/6848; G01N 2458/15

(57) ABSTRACT

Methods for measuring disialylated Fc glycan in a biological sample are described.

### CROSS-REFERENCE TO RELATED APPLICATIONS

This application claims the benefit of U.S. Provisional Application Ser. No. 63/116,643, filed on Nov. 20, 2020. The contents of the foregoing are incorporated herein by reference.

### SEQUENCE LISTING

The instant application contains a Sequence Listing which has been submitted electronically in ASCII format and is hereby incorporated by reference in its entirety. Said ASCII copy, created on Nov. 19, 2021, is named 14131_0237WO1_Seq_Listing.txt and is 33,641 bytes.

### BACKGROUND

Measurement of protein pharmacokinetics is important in both drug development and in treatment and monitoring of patients. In the case of antibody therapeutics, a sandwich ELISA specifically detects the unique portion of the antibody responsible for antigen binding (CDRs). Of course, such methods are not useful for measurement of therapeutics which are complex mixtures of antibodies.

### SUMMARY OF THE INVENTION

Described herein are methods for assessing the level of hypersialylated immunoglobulins (hsIgG) in a patient. The methods are useful for measuring the level of hsIgG after administration of a composition comprising hsIgG. The methods are also useful for assessing the level of naturally-occurring IgG antibodies, e.g., IgG1 antibodies, in a subject that are disialylated on both the α1,3 arm and the α1,6 arm of a branched glycan on the Fc domain of an IgG1 antibody. The methods entail the detection of a detectably labeled glycosylated peptide having the sequence EEQYNSTYR (SEQ ID NO: 1) wherein the N is glycosylated with A2F (EEQYNSTYR-A2F). The “A2F” glycan is also known as FA2G2S2 (Oxford Notation), and G2FS2 (short name used with IgG glycans), and is depicted by the following structure:

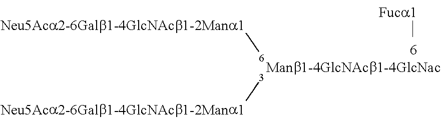

Described herein is a method for assessing a patient sample to determine the level of IgG1 that is disialylated on the Fc domain in the patient sample, the method comprising: providing a patient sample (e.g., a serum sample); adding a composition comprising detectably labeled EEQYNSTYR-A2F peptide to the sample; denaturing and trypsin digesting proteins in the sample to prepare a treated sample; subjecting treated sample to LC-MS/MS; and calculating the level of EEQYNSTYR-A2F peptide in the patient sample. In various embodiments: the patient has been administered a pharmaceutical composition comprising hsIgG; the step of calculating the EEQYNSTYR-A2F peptide in the patient sample comprises the use of a calibration curve generated using the pharmaceutical composition comprising hsIgG; the detectably labeled EEQYNSTYR-A2F is isotopically labeled; and greater than 80% of the EEQYNSTYR peptide in the composition comprising detectably labeled EEQYNSTYR-A2F is EEQYNSTYR-A2F.

Described herein are in vitro or ex vivo methods for assessing a patient sample to determine the level of IgG1 that is disialylated on the Fc domain in the patient sample, comprising: providing a patient sample; adding a composition comprising detectably labeled EEQYNSTYR-A2F peptide to the sample; denaturing and trypsin digesting proteins in the sample to prepare a treated sample; subjecting treated sample to LC-MS/MS; and calculating the level of EEQYNSTYR-A2F peptide in the patient sample.

In some embodiments, the detectably labeled EEQYNSTYR-A2F is isotopically labeled. In some embodiments, the detectably labeled EEQYNSTYR-A2F is isotopically labeled with Arg-10 (¹³C₆H₁₄15N₄O₂) and/or Lys-8 (¹³C₆H₁₄¹⁵N₂O₂). In some embodiments, the step of calculating the EEQYNSTYR-A2F peptide in the patient sample comprises the use of a calibration curve generated using the pharmaceutical composition comprising hsIgG. In some embodiments, the calibration curve is generated by plotting area ratio of the internal standard (IS) mass transition to the area of hsIgG mass transition. In some embodiments, the absolute abundance of hsIgG in the patient sample is determined using the calibration curve based on the area ratio for the unknown. In some embodiments, greater than 80% of the EEQYNSTYR peptide in the composition comprising detectably labeled EEQYNSTYR-A2F is EEQYNSTYR-A2F. In some embodiments, the patient has been administered a pharmaceutical composition comprising hsIgG.

Also described herein are compositions comprising: detectably labeled EEQYNSTYR-A2F; and a composition comprising disialylated IgG1.

Also described herein are in vitro or ex vivo methods for assessing a patient sample to determine the level of IgG2/3 that is disialylated on the Fc domain in the patient sample, comprising: providing a patient sample; adding a composition comprising detectably labeled EEQFNSTFR-A2F peptide to the sample; denaturing and trypsin digesting proteins in the sample to prepare a treated sample; subjecting treated sample to LC-MS/MS; and calculating the level of EEQFNSTFR-A2F peptide in the patient sample.

In some embodiments, the detectably labeled EEQFNSTFR-A2F is isotopically labeled. In some embodiments, the detectably labeled EEQFNSTFR-A2F is isotopically labeled with Arg-10 (¹³C₆H₁₄15N₄O₂) and/or Lys-8 (¹³C₆H₁₄¹⁵N₂O₂). In some embodiments, the step of calculating the EEQFNSTFR-A2F peptide in the patient sample comprises the use of a calibration curve generated using the pharmaceutical composition comprising hsIgG. In some embodiments, the calibration curve is generated by plotting area ratio of the internal standard (IS) mass transition to the area of hsIgG mass transition. In some embodiments, the absolute abundance of hsIgG in the patient sample is determined using the calibration curve based on the area ratio for the unknown. In some embodiments, greater than 80% of the EEQFNSTFR peptide in the composition comprising detectably labeled EEQFNSTFR-A2F is EEQFNSTFR-A2F. In some embodiments, the patient has been administered a pharmaceutical composition comprising hsIgG.

Also described herein are compositions comprising: detectably labeled EEQFNSTFR-A2F; and a composition comprising disialylated IgG.

Also described herein are in vitro or ex vivo methods for assessing a patient sample to determine the level of IgG3/4 that is disialylated on the Fc domain in the patient sample, comprising: providing a patient sample; adding a composition comprising detectably labeled EEQYNSTFR-A2F and/or EEQFNSTYR-A2F peptide to the sample; denaturing and trypsin digesting proteins in the sample to prepare a treated sample; subjecting treated sample to LC-MS/MS; and calculating the level of EEQYNSTFR-A2F and/or EEQFNSTYR-A2F peptide in the patient sample.

In some embodiments, the detectably labeled EEQYNSTFR-A2F and/or EEQFNSTYR-A2F is isotopically labeled. In some embodiments, the detectably labeled EEQYNSTFR-A2F and/or EEQFNSTYR-A2F is isotopically labeled with Arg-10 (¹³C₆H₁₄15N₄O₂) and/or Lys-8 (¹³C₆H₁₄¹⁵N₂O₂). In some embodiments, the step of calculating the EEQYNSTFR-A2F and/or EEQFNSTYR-A2F peptide in the patient sample comprises the use of a calibration curve generated using the pharmaceutical composition comprising hsIgG. In some embodiments, the calibration curve is generated by plotting area ratio of the internal standard (IS) mass transition to the area of hsIgG mass transition. In some embodiments, the absolute abundance of hsIgG in the patient sample is determined using the calibration curve based on the area ratio for the unknown. In some embodiments, greater than 80% of the EEQYNSTFR and/or EEQFNSTYR peptide in the composition comprising detectably labeled EEQYNSTFR-A2F and/or EEQFNSTYR-A2F. In some embodiments, the patient has been administered a pharmaceutical composition comprising hsIgG.

Also provided herein are compositions comprising: detectably labeled EEQYNSTFR-A2F and/or EEQFNSTYR-A2F; and a composition comprising disialylated IgG.

HsIgG, described in greater detail in WO2020/215021, WO/2014/018747, WO/2014/179601 and WO/2015/05762 has a very high level of sialic acid on the branched glycans on the Fc region of the immunoglobulins, for example, at least 50% (60%, 70%, 80%, 90% or more) of the branched glycans on the Fc region of the immunoglobulins are sialylated via NeuAc-α2,6-Gal terminal linkages on both the α1,3 arm and the α1,6 arm of the branched glycan. HsIgG contains a diverse mixture of IgG antibodies, primarily IgG1 antibodies. The diversity of the antibodies is high. The immunoglobulins used to prepare hsIgG can be obtained, for example from pooled human plasma (e.g., pooled plasma from at least 1,000-30,000 donors). Alternatively, IVIG can be used to prepare hsIgG. In hsIgG at least 50% (e.g., 60%, 70%, 80%, 82%, 85%, 87%, 90%, 92%, 94%, 95%, 97%, 98% up to and including 100%) of branched glycans on the Fc region of the immunoglobulins have a sialic acid residue on both the α1,3 arm and the α1,6 arm (i.e., are disialylated by way of NeuAc-α2,6-Gal terminal linkages). In some embodiments, in addition to the Fc sialylation, at least 50% (e.g., 60%, 70%, 80%, 82%, 85%, 87%, 90%, 92%, 94%, 95%, 97%, 98% or up to and including 100%) of branched glycans on the Fab region are disialylated by way of NeuAc-α2,6-Gal terminal linkages. In some cases, at least 85%, (87%, 90%, 92%, 94%, 95%, 97%, 98% or up to and including 100%) of total branched glycans (sum of glycans on the Fc and Fab domains) are disialylated by way of NeuAc-α2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (e.g., sialylated only on the α1,3 arm or the α1,6 arm) by way of a NeuAc-α2,6-Gal terminal linkage. HsIgG preparations are primarily IgG antibodies (e.g., at least 80%, 85%, 90%, 95% wt/wt of the immunoglobulins are IgG antibodies of various isotypes).

As used herein, the term “Fc region” refers to a dimer of two “Fc polypeptides,” each “Fc polypeptide” including the constant region of an antibody excluding the CH1 domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains.

As used herein, “glycan” is a sugar, which can be monomers or polymers of sugar residues, such as three or more sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

IVIg is a preparation of pooled, polyvalent immunoglobulins, including all four IgG isotypes, extracted from plasma of at least 1,000 human donors. Among the forms of IVIg approved for use in the United States are Gammagard (Baxter Healthcare Corporation), Gammaplex (Bio Products Laboratory), Bivigam (Biotest Pharmaceuticals Corporation), Carimmune NF (CSL Behring AG), Gamunes-C (Grifols Therapeutics, Inc.) Glebogamma DID (Instituto Grifols, SA) and Octagam (Octapharma Pharmazeutika Produktionsges Mbh). IVIg is approved as a plasma protein replacement therapy for immune deficient patients and for other uses. The level of IVIg Fc glycan sialylation varies among IVIg preparations, but is generally less than 20%. The level of disialylation is generally far lower.

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal sialyltransferase and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 arm or an α1,6 arm. The term “disialylated” refers to a branched glycan having a terminal sialic acid on two arms, e.g., both an α1,3 arm and an α1,6 arm.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 Left panel: Schematic representation of enzymatic sialylation reaction to transform pooled immunoglobulins to hsIgG. Right panel: IgG Fc glycan profile for the starting IVIg (upper) and for hsIgG (lower) enzymatically prepared from IVIg. Glycan profiles for the different IgG subclasses are derived via glycopeptide mass spectrometry analysis. Peptide sequences used to quantify glycopeptides for different IgG subclasses are: IgG1=EEQYNSTYR (SEQ ID NO: 1), IgG2/3 EEQFNSTFR (SEQ ID NO: 2), IgG3/4 EEQYNSTFR (SEQ ID NO: 3) and EEQFNSTYR (SEQ ID NO: 4). Bars, from left to right: IgG1, IgG2/3, IgG3/4.

FIG. 2 Example of chromatographic method. Upper panels: LLOQ sample (5.0 ug/mol in 5% BSA PBS). Lower panels: plasma sample (20 ug/ml).

FIG. 3: Depicts relevant mass transitions for the EEQYNSTYR-A2 peptide.

### DETAILED DESCRIPTION

Immunoglobulins are glycosylated at conserved positions in the constant regions of their heavy chain. For example, human IgG has a single N-linked glycosylation site at Asn297 of the CH2 domain. Each immunoglobulin type has a distinct variety of N-linked carbohydrate structures in the constant regions. For human IgG, the core oligosaccharide normally consists of GlcNAc₂Man₃GlcNAc, with differing numbers of outer residues. Variation among individual IgG's can occur via attachment of galactose and/or galactose-sialic acid at one or both terminal GlcNAc or via attachment of a third GlcNAc arm (bisecting GlcNAc).

The present disclosure encompasses, in part, pharmaceutical preparations including pooled human immunoglobulins having an Fc region having particular levels of branched glycans that are sialylated on both of the branched glycans in the Fc region (e.g., with a NeuAc-α2,6-Gal terminal linkage).

Preparations of pooled, polyvalent human immunoglobulins, including IVIg preparations, are highly complex because they are highly heterogeneous in several regards. They include immunoglobulins pooled from many hundreds or more than 1000 individuals. While at least about 90% or 95% of immunoglobulins are IgG isotype (of all subclasses), other isotypes, including IgA and IgM are present. The immunoglobulins in IVIg and preparations of pooled, polyvalent human immunoglobulins vary in both specificity and glycosylation pattern.

Hypersialylation of pooled, polyvalent immunoglobulins alters the glycans which are present on the immunoglobulins. For some glycans, the alteration entails the addition of one of more galactose molecules and the addition of one or more sialic acid molecules. For other glycans, the alteration entails only the addition of one or more sialic acid molecules. Moreover, while essentially all IgG antibodies, the predominant immunoglobulins in preparations of pooled, polyvalent immunoglobulins, have a glycosylation site on each polypeptide forming Fc region, not all IgG antibodies have a glycosylation site on the Fab domain. Altering the glycosylation of an immunoglobulin preparation alters the structure and activity of the individual immunoglobulins in the preparation and, importantly, alters the interactions between individual immunoglobulins as well as the bulk behavior of preparations of the immunoglobulins.

The widely used formulation used for IVIg preparations is wholly unsuitable for pharmaceutical preparations of hypersialylated immunoglobulins (hsIgG) for at least the reason that the formulations, when used for hsIgG, are not stable to shear stress that occurs in normal shipping of pharmaceutical formulations. When subjected to this type of shear stress, subvisible particles formed in the hsIgG formulations. It is known that such subvisible particles in antibody preparations can cause serious adverse events at the site of injection and off target immune responses. Subvisible particles in antibody preparations can also activate the complement system, cause embolisms, and other negative immunogenic reactions. It was found that the addition of non-ionic surfactants rendered the hsIgG preparations more stable to shear stress and greatly reduced the formation of subvisible particles.

Due to their highly complex and heterogenous nature, methods for measuring amounts of monoclonal antibodies in a patient cannot be used to measure amounts of hsIgGs in a patient. The present disclosure encompasses, in part, methods for determining the level of hsIgGs in a patient. These methods can be used, for example, to monitor the levels after administration of a pharmaceutical composition of hsIgG or to measure the naturally-occurring levels of IgG in a patient. The information provided by the methods described herein may be used, for example, to diagnose a patient, to monitor treatment of a patient, to monitor naturally-occurring levels of hsIgG in a patient, to monitor levels of hsIgG in a patient and then administer treatment, or to adjust levels of a pharmaceutical composition administered to a patient, etc.

Naturally derived polypeptides that can be used to prepare hsIgG include, for example, immunoglobulins isolated from pooled human serum. HsIgG can also be prepared from IVIg and polypeptides derived from IVIg. HsIgG can be prepared as described in WO2014/179601. Preparation of hsIgG is also described in Washburn et al. (Proc Natl Acad Sci USA 2015 Mar. 17;112 (11):E1297-306).The level of sialylation in a hsIgG preparation can be measured on the Fc domain (e.g., the number of branched glycans that are sialylated on an α1,3 arm, an α1,6 arm, or both, of the branched glycans in the Fc domain), or on the overall sialylation (e.g., the number or percentage of branched glycans that are sialylated on an α1,3 arm, an α1,6 arm, or both, of the branched glycans in the preparation of polypeptides whether on the Fc domain or the Fab domain).

Is some cases, the pooled serum used as a source of immunoglobulins for preparing hsIgG is isolated from a specific population of individuals, for example, individuals that produce antibodies against one or more virus, such as COVID-19, SARS, parainfluenza, influenza, but do not have an active infection. In some cases, the immunoglobulins are isolated from a population of individuals in which greater than 50%, 55%, 60%, 75% produce antibodies to a selected virus.

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α1,3 arm,” and the second branch is referred to as the “α1,6 arm,” as shown below. Yellow circles are Gal; green circles are Man; triangles are Fuc, diamonds are NANA; squares are GlcNAc.

N-glycans can be subdivided into three distinct groups called “high mannose type,” “hybrid type,” and “complex type,” with a common pentasaccharide core (Man(α1,6)-(Man(α1,3))-Man(β1,4)-GlcpNAc(β1,4)-GlcpNAc(β1,N)-Asn) occurring in all three groups.

After initial processing in the endoplasmic reticulum, the polypeptide is transported to the Golgi where further processing may take place. If the glycan is transferred to the Golgi before it is completely trimmed to the core pentasaccharide structure, it results in a “high-mannose glycan.”

Additionally or alternatively, one or more monosaccharies units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α2,3 or α2,6.

Fc regions are glycosylated at conserved, N-linked glycosylation sites. For example, each heavy chain of an IgG antibody has a single N-linked glycosylation site at Asn297 of the C_{H}2 domain. IgA antibodies have N-linked glycosylation sites within the C_{H}2 and C_{H}3 domains, IgE antibodies have N-linked glycosylation sites within the C_{H}3 domain, and IgM antibodies have N-linked glycosylation sites within the C_{H}1, C_{H}2, C_{H}3, and C_{H}4 domains.

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR. For human IgG, the core oligosaccharide normally consists of GlcNAc₂Man₃GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc). Glycans of polypeptides can be evaluated using any methods known in the art. For example, sialylation of glycan compositions (e.g., level of branched glycans that are sialylated on an α1,3 arm and/or an α1,6 arm) can be characterized using methods described in WO2014/179601.

### Example 1: Hypersialylated IgG

Hypersialylated IgG in which more than 60% of the branched Fc region glycans are disialylated was prepared as generally described in WO2014/179601.

Briefly, IVIg is exposed to a one-pot sequential enzymatic reaction using β1,4 galactosyltransferase 1 (B4-GalT) and α2,6-sialyltransferase (ST6-Gal1) enzymes. The galactosyltransferase enzyme selectively adds galactose residues to pre-existing asparagine-linked glycans in IVIg. The resulting galactosylated glycans serve as substrates to the sialic acid transferase enzyme which selectively adds sialic acid residues to cap the asparagine-linked glycan structures attached to IVIg. Thus, the overall sialylation reaction employed two sugar nucleotides (UDPGal and CMP-NANA). The latter was replenished periodically to increase di-sialylated product relative to monosialylated product. The reaction includes the co-factor manganese chloride.

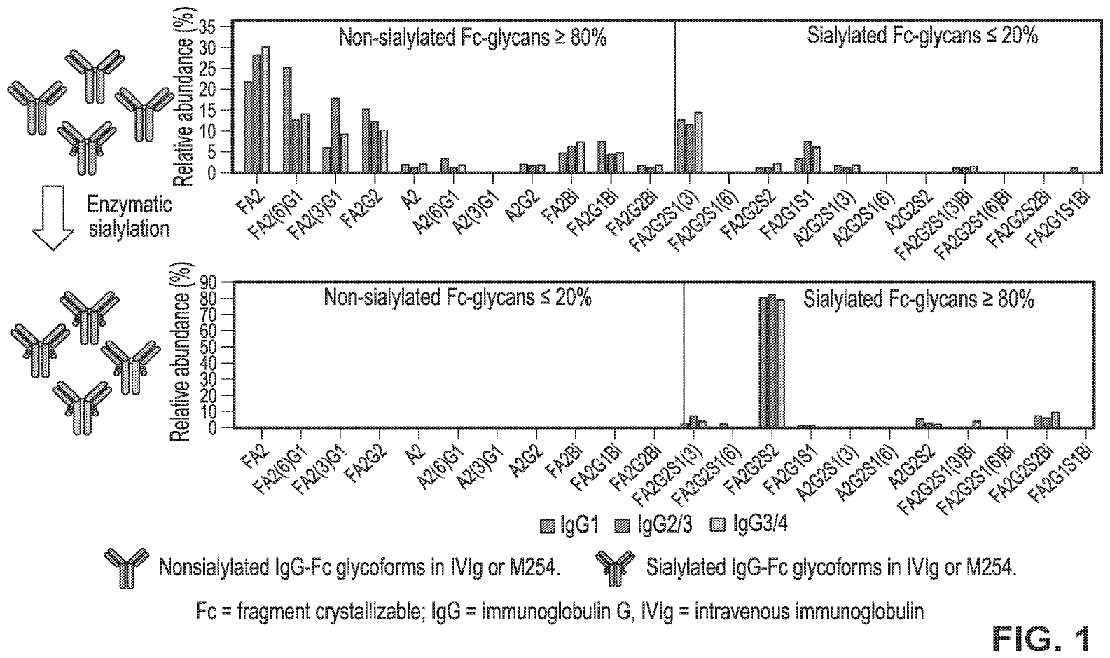

A representative example of the corresponding IgG-Fc glycan profile for the starting IVIg and the reaction product is shown in FIG. 1. The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is <20%. For the reaction product, the sum for all nonsialylated glycans is <20% and the sum for all sialylated glycans is more than 80%. Nomenclature for different glycans listed in the glycoprofile use the Oxford notation for N linked glycans.

### Example 2: Quantification of HsIgG

A highly sialylated IgG1 Fc domain was prepared. Briefly, recombinant IgG1 Fc domain was produced in HEK cell grown in arginine (Arg) and lysine (Lys) free media supplemented with Arg-10 (¹³C₆H₁₄15N₄O₂) and Lys-8 (¹³C₆H₁₄^{N}₂O₂) and subsequently purified. The purified, isotopically labeled Fc domain is then enzymatically galactosylated and sialylated and used as an internal standard. One suitable method for galactosylation and sialylation is that described in Washburn et al. (Proc Natl Acad Sci USA 2015 Mar. 17;112 (11):E1297-306). Alternatively, isotopically labeled IgG1 Fc domain is buffer exchanged into 50 mM BIS-TRIS/150 mM NaCl pH 6.9. The recombinant Fc (4.5 mL of 55 mg/mL) is galactosylated by addition of 158 μL of 1 M of MnCl₂, 121 μL of 1 M UDP-Gal, and 76 μL of B4-GalT1 enzyme 5.9 mg/ml). The sample is incubated at 37° C. for 19 hours. Next, 645 μL of ST6 (13 mg/ml or about 42 U/ml ) and 95 μL of 1M CMP-NANA is added and the sample is incubated at 37° C. for 9 hours. Next, a second aliquot (95 μL) of 1M CMP-NANA is added and the sample is again incubated at 37° C. A third aliquot of CMP-NANA is added 23.75 hours after the first aliquot (about 14.75 hours after the second aliquot). Next, 2 mL of EDTA is added and the sample was placed at 4° C. for 32.5 hours. The sample centrifuged and the supernatant is decanted and sterile filtered using 0.2 μm Vivaspin 6 filter. The sample is then applied to a Protein A column that has been washed with 0.1N NaOH, guanidine HCl, and 1×PBS. After loading, the column is washed with 1×PBS, 5×PBS, and then 1×PBS. The desired material is eluted with 100 mM pH 3.0 glycine buffer, nutralized with 1/10 volume 1 M TRIS pH 8.8 buffer, buffer exchanged into 1×PBS and sterile filtered.

The resulting IgG1Fc domain (labeled disialylated Fc domain) is greater than 80% disialylated on the branched glycans.

To create a calibration curve for assessing hsIgG, e.g., hsIgG in a patient treated with a hsIgG composition, the labeled disialylated Fc domain is spiked into different concentrations of the hsIgG composition in a suitable biological matrix (e.g., 2% BSA in phosphate buffered saline). The samples are denatured, digested with trypsin, cleaned up and analyzed by LC-MS/MS. The glycosylated peptide measured is EEQYNSTYR modified at the N with A2F (“EEQYNSTYR-A2F)”. The “A2F” glycan is also known as FA2G2S2 (Oxford Notation), and G2FS2 (short name used with IgG glycans), and is depicted by the following structure:

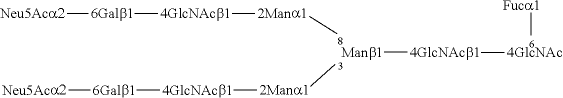

| Species | Peptide | Represents | Q1 | Q3-1 |
|---|---|---|---|---|
| 1 | EEQYNSTYR-2F | hslgG1 | 1181.3 | 1443.1 |
| 2 | EEQYNSTYR*-2F | hslgG1 Internal Standard | 1184.4 | 1447.8 |

The EEQYNSTYR-2F peptide is specific for IgG1 Fc that is disialylated on branched glycans. Other peptides can be used to assess other IgG subclasses. For IgG2/3 antibodies, EEQFNSTFR (SEQ ID NO: 2) modified at the N with A2F (EEQFNSTFR-A2F) can be used. For IgG3/4 EEQYNSTFR (SEQ ID NO: 3) and EEQFNSTYR (SEQ ID NO: 4) modified at the N with A2F (EEQYNSTFR-A2F and EEQFNSTYR-A2F, respectively) can be used.

A calibration curve is generated by plotting area ratio of the internal standard (IS) mass transition to the area of the hsIgG mass transition. The absolute abundance of hsIgG in a biological matrix (e.g., a patient serum sample) is determined using this calibration curve based on the area ratio for the unknown.

Suitable sample preparation conditions for analysis of a patient plasma sample include: add 10 μL labeled, disialylated Fc domain (internal standard) in working solution to 25 μL plasma; add 25 μL 50 mM ammonium bicarbonate+50 μL 2% sodium deoxycholate; incubate at 75° C. for 45 min to denature proteins; spin down and collect pellet; resuspend pellet in 200 μL 0.5 mg/mL trypsin in 50 mM ammonium bicarbonate; incubate at 37° C. for 180 min; add 50 μL 10% trifluoroacetic acid to stop digestion and precipitate deoxycholate; remove deoxcholate by centrifugation.

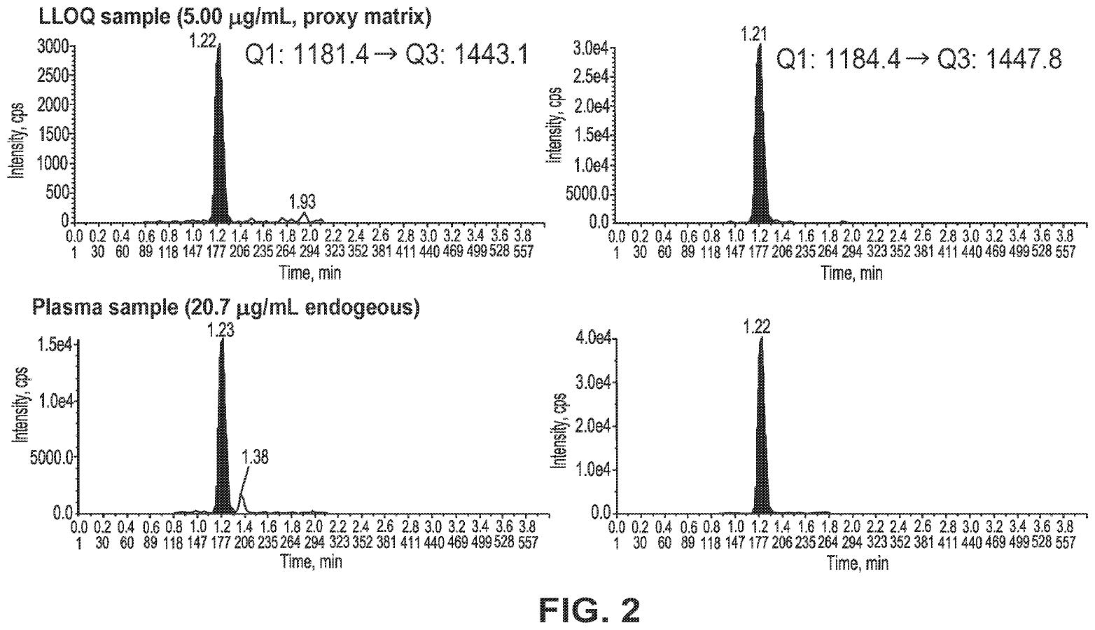

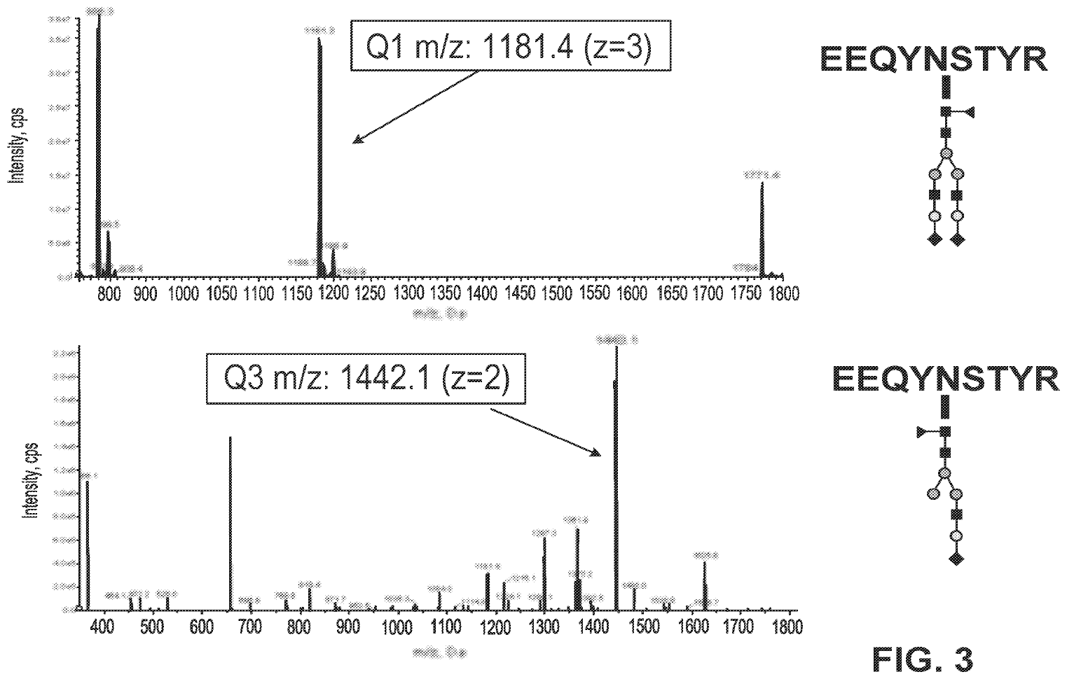

Analysis by LC-MS/MS can employ an Acquity CSH C18 (2.1 mm×100 mm, 1.7 μm particles) column (Waters, Inc.) (mobile phase A=10% methanol/0.1% formic acid in water; mobile phase B=acetonitrile) and Sciex triple quad 6500+ System. FIG. 2 depicts an example of the results of this chromatographic method and FIG. 3 depicts an example of the observed mass transitions.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An in vitro or ex vivo method for assessing a patient sample to determine the level of IgG1 that is disialylated on the Fc domain in the patient sample, the method comprising:
providing a patient sample;
adding a composition comprising detectably labeled EEQYNSTYR-A2F peptide to the sample;
denaturing and trypsin digesting proteins in the sample to prepare a treated sample;
subjecting treated sample to LC-MS/MS; and
calculating the level of EEQYNSTYR-A2F peptide in the patient sample.

2. (canceled)

3. (canceled)

4. The method of claim 1, wherein the step of calculating the EEQYNSTYR-A2F peptide in the patient sample comprises the use of a calibration curve generated using the pharmaceutical composition comprising hsIgG.

5. (canceled)

6. (canceled)

7. The method of claim 1, wherein greater than 80% of the EEQYNSTYR peptide in the composition comprising detectably labeled EEQYNSTYR-A2F is EEQYNSTYR-A2F.

8. The method of claim 1, wherein the patient has been administered a pharmaceutical composition comprising hsIgG.

9. (canceled)

10. An in vitro or ex vivo method for assessing a patient sample to determine the level of IgG2/3 that is disialylated on the Fc domain in the patient sample, the method comprising:
providing a patient sample;
adding a composition comprising detectably labeled EEQFNSTFR-A2F peptide to the sample;
denaturing and trypsin digesting proteins in the sample to prepare a treated sample;
subjecting treated sample to LC-MS/MS; and
calculating the level of EEQFNSTFR-A2F peptide in the patient sample.

11. The method of claim 10, wherein the detectably labeled EEQFNSTFR-A2F is isotopically labeled.

12. (canceled)

13. The method of claim 10, wherein the step of calculating the EEQFNSTFR-A2F peptide in the patient sample comprises the use of a calibration curve generated using the pharmaceutical composition comprising hsIgG.

14. The method of claim 13, wherein the calibration curve is generated by plotting area ratio of the internal standard (IS) mass transition to the area of hsIgG mass transition.

15. The method of claim 13, wherein the absolute abundance of hsIgG in the patient sample is determined using the calibration curve based on the area ratio for the unknown.

16. The method of claim 10, wherein greater than 80% of the EEQFNSTFR peptide in the composition comprising detectably labeled EEQFNSTFR-A2F is EEQFNSTFR-A2F.

17. The method of claim 10, wherein the patient has been administered a pharmaceutical composition comprising hsIgG.

18. (canceled)

19. An in vitro or ex vivo method for assessing a patient sample to determine the level of IgG3/4 that is disialylated on the Fc domain in the patient sample, the method comprising:
providing a patient sample;
adding a composition comprising detectably labeled EEQYNSTFR-A2F and/or EEQFNSTYR-A2F peptide to the sample;
denaturing and trypsin digesting proteins in the sample to prepare a treated sample;
subjecting treated sample to LC-MS/MS; and
calculating the level of EEQYNSTFR-A2F and/or EEQFNSTYR-A2F peptide in the patient sample.

20. The method of claim 19, wherein the detectably labeled EEQYNSTFR-A2F and/or EEQFNSTYR-A2F is isotopically labeled.

21. The method of claim 20, wherein the detectably labeled EEQYNSTFR-A2F and/or EEQFNSTYR-A2F is isotopically labeled with Arg-10 (¹³C₆H₁₄15N₄O₂) and/or Lys-8 (¹³C₆H₁₄¹⁵N₂O₂).

22. The method of claim 19 21, wherein the step of calculating the EEQYNSTFR-A2F and/or EEQFNSTYR-A2F peptide in the patient sample comprises the use of a calibration curve generated using the pharmaceutical composition comprising hsIgG.

23. The method of claim 22, wherein the calibration curve is generated by plotting area ratio of the internal standard (IS) mass transition to the area of hsIgG mass transition.

24. The method of claim 22, wherein the absolute abundance of hsIgG in the patient sample is determined using the calibration curve based on the area ratio for the unknown.

25. The method of claim 19, wherein greater than 80% of the EEQYNSTFR and/or EEQFNSTYR peptide in the composition comprising detectably labeled EEQYNSTFR-A2F and/or EEQFNSTYR-A2F.

26. The method claim 19, wherein the patient has been administered a pharmaceutical composition comprising hsIgG.

27. A composition comprising:
detectably labeled EEQYNSTFR-A2F and/or EEQFNSTYR-A2F.
